(19) United States
(12) Patent Application Publication
(10) Pub. No.: US 2003/0187414 A1
(43) Pub. Date: Oct. 2, 2003

(54) Disposable diaper and method therefor

(76) Inventors: Edward Reiss, Westport, CT (US); Brenda J. Schenk, Westport, CT (US)

Correspondence Address: WEISS & MOY PC, 4204 NORTH BROWN AVENUE, SCOTTSDALE, AZ, 85251, US

(21) Appl. No.: 10/109,091

(22) Filed: Mar. 27, 2002

(51) Int. Cl. A61F 13/15; A61F 13/20

(52) U.S. Cl. 604/367; 604/375

(57) ABSTRACT

A disposable diaper has a multi-layered diaper assembly. The disposable diaper has a first top surface exterior layer assembly comprising a non-woven material having a planar, soft, cloth-like surface layer. A second layer is provided and consists of non-chlorine bleached wood pulp. A third layer is provided having a top surface portion in contact with a bottom surface portion of the second layer. A fourth bottom surface interior layer is provided in contact with a bottom surface portion of the third layer and comprising a non-woven liquid permeable material having a planar, soft, cloth-like body contact surface portion.

### RELATED APPLICATIONS

The present application is related to U.S. Pat. No. 5,743,895 entitled “DISPOSABLE DIAPER AND METHOD THEREFOR”, issued on Apr. 28, 1998 in the name of the same inventors and incorporated by reference into the present application.

### BACKGROUND OF THE INVENTION

1. Field of the Invention

This invention relates to a disposable diaper and, more specifically, to a disposable diaper and method therefor that uses more natural materials in the manufacturing process.

2. Description of the Prior Art

Disposable diapers are well known and widely used. Over the years, disposable diapers have become the dominant diaper, largely replacing the old form of reusable diaper. Typical disposable diapers generally use an absorbent interior area for absorbing a wearer's urine, bounded by a thin plastic exterior layer. The interior portion of the typical disposable diaper presently on the market is generally comprised of a bottom layer of non-woven material that is in direct contact with the area of the child's skin covered by the diaper, a backing layer of tissue paper that contacts the bottom portion of the layer of non-woven material, a wadding batt layer of wood pulp that is located between the thin plastic exterior layer and the layer of tissue paper, and, in order to increase the diaper's fluid absorbency, diaper manufacturers add a superabsorbent polymer or other chemical additive to the wadding batt layer. Without the superabsorbent polymer, wood pulp generally absorbs in the range of 12 to 15 times its weight. With the superabsorbent polymer, the wadding batt layer is able to absorb in the range of 30 to 55 times its weight.

One problem with current disposable diapers is that the wood pulp used is bleached with chlorine. This bleaching process produces low levels of dioxins or furons as by-products. While the government may allow certain low levels of dioxins, dioxins are toxic and carcinogenic.

Another problem with current disposable diapers is that in order to increase fluid absorbency, a superabsorbent material is added to the wadding batt layer. In present disposable diapers, the superabsorbent material is an artificial superabsorbent polymer. Diapers with this polymer additive experience what is known in the industry as “rewet”—i.e., the transfer of wetness from the diaper back to the surface of the diaper and to the child's skin.

Therefore, a need existed to provide an improved disposable diaper and method. The improved disposable diaper and method must use more natural materials. The improved disposable diaper and method must use materials that do not use a chlorine bleach during the manufacturing process. The improved disposable diaper and method may further use a more natural superabsorbent mixture.

### SUMMARY OF THE INVENTION

In accordance with one embodiment of the present invention, it is an object of the present invention to provide an improved disposable diaper and method.

It is another object of the present invention to provide an improved disposable diaper and method that overcomes the above problems associated with prior art disposable diapers.

It is still another object of the present invention to provide an improved disposable diaper and method that uses more natural materials.

It is still another object of the present invention to provide an improved disposable diaper and method that use materials that do not use a chlorine bleach during the manufacturing process.

It is yet another object of the present invention to provide an improved disposable diaper and method that may further use a more natural superabsorbent mixture.

### BRIEF DESCRIPTION OF THE EMBODIMENTS

In accordance with one embodiment of the present invention a disposable diaper is disclosed. The disposable diaper has a multi-layered diaper assembly. The disposable diaper has a first top surface exterior layer assembly comprising a non-woven material having a planar, soft, cloth-like surface layer. A second layer is provided and consists of non-chlorine bleached wood pulp. A third layer is provided having a top surface portion in contact with a bottom surface portion of the second layer. A fourth bottom surface interior layer is provided in contact with a bottom surface portion of the third layer and comprising a non-woven liquid permeable material having a planar, soft, cloth-like body contact surface portion.

In accordance with another embodiment of the present invention, a method for providing a disposable diaper is disclosed. The method comprises the following steps: forming a first top surface exterior layer assembly comprising a non-woven material having a planar, soft, cloth-like surface layer; forming a second layer consisting solely of non-chlorine bleached wood pulp; providing a third layer comprising a thin planar layer of tissue paper having a top surface portion in contact with a bottom surface portion of the second layer; and providing a fourth bottom surface interior layer in contact with a bottom surface portion of the third layer and comprising a non-woven liquid permeable material having a planar, soft, cloth-like, body contact surface portion.

The foregoing and other objects, features, and advantages of the invention will be apparent from the following, more particular, description of the preferred embodiments of the invention, as illustrated in the accompanying drawing.

### BRIEF DESCRIPTION OF THE DRAWINGS

The novel features believed characteristic of the invention are set forth in the appended claims. The invention itself, as well as a preferred mode of use, and advantages thereof, will best be understood by reference to the following detailed description of illustrated embodiments when read in conjunction with the accompanying drawings.

FIG. 1 is an elevated front side view of one embodiment of the disposable diaper of the present invention.

FIG. 2 is an elevated rear side or backside view of one embodiment the disposable diaper of the present invention.

FIG. 3 is a cross-sectional view of the disposable diaper of FIG. 1 taken along line 3—3 of FIG. 1.

### DETAILED DESCRIPTION OF THE PREFERRED EMBODIMENT

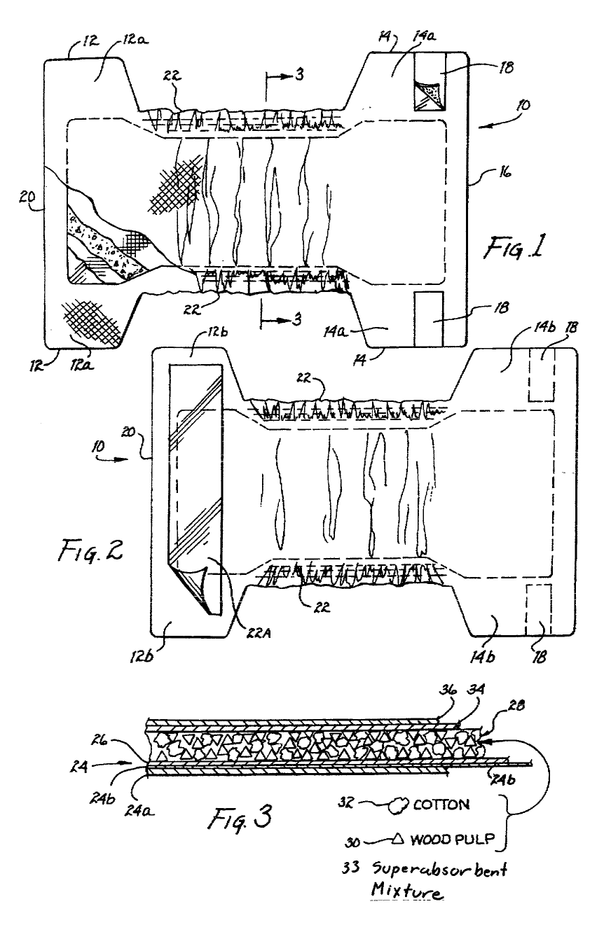

Referring to the embodiment of FIGS. 1-3, reference number 10 refers generally to the disposable diaper of this invention. It should be noted that the following description is one embodiment of the present invention.

Referring to FIG. 1, a front or interior view of the disposable diaper 10 is shown, in the open position. The disposable diaper 10, like other disposable diapers, comprises front flaps 12 and back flaps 14. Reference numbers 12a and 14a refer to the side portions of the front flaps 12 and the back flaps 14, respectively, that are visible in the front view of the disposable diaper 10 depicted in FIG. 1. Adhered to the back flaps 14a, and oriented substantially parallel to an upper edge 16 of the diaper 10, are preferably two tape assemblies 18. Each tape assembly 18 comprises a base portion that is attached directly to the surface of the diaper 10, and a partially separable adhesive portion that may be peeled from the base portion for adhesion attachment to the reverse side of front flaps 12 (see both FIGS. 1 and 2) when the diaper 10 is being placed on a baby or child. On both sides of the diaper 10, oriented in a substantially perpendicular direction to the upper edge 16 and the lower edge 20, are two elastic portions 22. The elastic portions 22 snugly grip the legs of the child during the wearing of the diaper 10 to, among other things, minimize the leaking of fluid out of the diaper 10.

Referring to FIG. 2, a rear or backside exterior view of the disposable diaper 10 is shown, in the open position. Reference numbers 12b and 14b refer to the sides of the front flaps 12 and the back flaps 14, respectively, that are visible in the rear or backside view of the disposable diaper 10. Adhered to the back flaps 14b, and oriented substantially parallel to the upper edge 16 of the diaper 10, are portions of the two tape assemblies 18. These are continuations of the portions of the tape assemblies 18 which are located on the back flaps 14a, as shown in FIG. 1. Located between tabs 12b is a preferably rectangular plastic strip 22A, which strip 22A receives the adhesive portion of the tape assemblies 18 when the diaper is being placed on a baby or child.

Referring to FIG. 3, a cross-sectional view of the disposable diaper 10 is shown, showing the layers of material comprising the diaper 10. The interior portion of the disposable diaper 10 is comprised of a bottom layer 24a of non-woven material that is in direct contact with the area of the child's skin covered by the disposable diaper 10. A backing layer 26 contacts the bottom portion of the layer of non-woven material. The backing layer 26 may be tissue paper or the like. It should be noted that the description of the different types of materials for the backing layer 26 should not be seen as to limit the scope of the present invention. In accordance with one embodiment of the present invention, an underlying thin plastic sheet 24b is provided, with the surface layer 24a and the plastic sheet 24b being in bonded contact with each other, preferably in the form of a laminate.

In contact with a bottom or inner portion of the tissue layer 26 or, if desired, directly in contact with a bottom or inner surface portion of the plastic sheet 24b, is a second layer 28.

The second layer 28 is a wadding batt layer consisting of a wood pulp mixture 30. All present disposable diapers bleach their wood pulp with chlorine. This bleaching process low levels of dioxins. Even though low levels of dioxins are accepted by the government, many people would prefer to have a disposable diaper 10 made from non-chlorine bleached materials. Thus, in accordance with one embodiment of the present invention, a non-chlorine bleached wood pulp 30 is used. A bleached untreated softwood fluff pulp which is biodegradable and totally chlorine free may also be used. Additionally, wood pulp 30 used in the present invention may be bleached using other processes other than chlorine. In accordance with one embodiment of the present invention, the wood pulp 30 is bleached with a hydrogen peroxide mixture. The use of hydrogen peroxide should not be seen as to limit the scope of the present invention.

The second layer 28 may be comprised solely of non-chlorine bleached wood pulp 30. However, the non-chlorine bleached wood pulp 30 may be combined with other materials. For example, cotton 32 may be combined with the wood pulp 30. The cotton 32 may be a regular (non-organic) cotton 32, a certified organic cotton 32, or combinations thereof. The wood pulp 30 and cotton 32 mixture creates a blend that is substantially more absorbent than a wadding batt layer comprised entirely of wood pulp 30. In this regard, wood pulp 30 generally absorbs in the range of 12 to 15 times its weight. Cotton pulp 32, in contrast, generally absorbs approximately 25 times its weight, depending on the quality of the particular cotton. By mixing cotton pulp 32 with wood pulp 30 in appropriate amounts, the absorbency of the resulting mixture is greater than that of pure wood pulp 30. In the preferred embodiment of this invention, the second layer 28 is about five-eighths of an inch thick. However, it should be understood that changes can be made in the thicknesses of any of the layers comprising the diaper 10 without departing from the spirit or scope of this invention.

The second layer 28 may further comprise a superabsorbent mixture 33. The superabsorbent mixture will allow the wadding batt layer to absorb in the range of 30 to 55 times its weight. However, instead of a superabsorbent polymer such as polyacrylates, one that is more friendly to the skin would be used. The superabsorbent mixture may be a natural or an artificial superabsorbent mixture. In accordance with one embodiment of the present invention, a natural wheat based polysaccharide superabsorbent mixture is used. This type of superabsorbent mixture is biodegradable and is friendlier to the skin of the wearer and changer. It should be noted that the wheat based polysaccharide superabsorbent mixture is just an example and should not be seen as to limit the scope of the present invention. The superabsorbent mixture will still allow the wadding batt layer to absorb in the range of 30 to 55 times it weight, but will not have the side effects of irritating the wearer's skin during “rewet”.

In contact with a bottom or upper portion of second layer 28 is a third layer 34, comprising tissue paper. The tissue paper may be a regular type of tissue paper or a non-chlorine bleached tissue paper. The tissue paper would be bleached using a process similar to that described above. Finally, in contact with a bottom or upper portion of third layer 34 is a fourth layer 36, comprising a non-woven bond material such as a polyester type of material. Accordingly, the fourth layer 36 is made of a soft cloth-like type material that is substantially porous, permitting a child's or baby's fluid waste to penetrate therethrough for fluid absorption by the second layer 28, whereas the first layer 24 provides both a barrier layer to prevent fluid from penetrating outwardly therethrough from the second fluid absorbing layer 28 and a soft, cloth-like surface layer (24a) to provide a soft exterior layer that is non-abrasive to the skin of a baby or child or a person diapering the baby or child. Thus, both inner and outer soft layers are provided by this invention.

While the invention has been particularly shown and described with reference to preferred embodiments thereof, it will be understood by those skilled in the art that the foregoing and other changes in form and details may be made therein without departing from the spirit and scope of the invention.

What is claimed is:

1. A disposable diaper comprising, in combinations:
a multi-layered diaper assembly comprising:
a first top surface exterior layer assembly comprising a non-woven material having a planar, soft, cloth-like surface layer, the non-woven material surface layer;
a second layer consisting of non-chlorine bleached wood pulp;
a third layer having a top surface portion in contact with a bottom surface portion of the second layer; and
a fourth bottom surface interior layer in contact with a bottom surface portion of the third layer and comprising a non-woven liquid permeable material having a planar, soft, cloth-like body contact surface portion.

2. A disposable diaper in accordance with claim 1 wherein the second layer further comprises a blended mixture of non-chlorine bleached wood pulp and cotton pulp having a substantially higher liquid urine absorption than wood pulp and located below a bottom surface of the underlying thin plastic sheet.

3. A disposable diaper in accordance with claim 1 wherein the second layer further comprises a suberabsorbent mixture.

4. A disposable diaper in accordance with claim 3 wherein the suberabsorbent mixture is a natural suberabsorbent mixture.

5. A disposable diaper in accordance with claim 4 wherein the natural suberabsorbent mixture is a wheat based polysaccharide suberabsorbent mixture.

6. A disposable diaper in accordance with claim 1 wherein the second layer further comprises a blended mixture of non-chlorine bleached wood pulp, cotton pulp, and a wheat based superabsorbent mixture located below the bottom surface of the underlying thin plastic sheet.

7. A disposable diaper in accordance with claim 1 wherein the first top surface exterior layer assembly further comprises:
an underlying thin, liquid impermeable plastic sheet;
a layer of tissue paper having a top surface thereof in contact with a bottom surface of the underlying thin plastic sheet and a bottom surface thereof in contact with a top surface of the second layer.

8. A disposable diaper in accordance with claim 7 wherein the layer of tissue paper is non-chlorine bleached tissue paper.

9. A disposable diaper in accordance with claim 1 wherein the first layer is about 2 mils thick and the second layer is about five-eighths of an inch thick.

10. A method for providing a multi-layered disposable diaper comprising the following steps:
forming a first top surface exterior layer assembly comprising a non-woven material having a planar, soft, cloth-like surface layer, the non-woven material surface layer being coupled to a backing sheet;
forming a second layer consisting solely of non-chlorine bleached wood pulp;
providing a third layer comprising a thin planar layer of tissue paper having a top surface portion in contact with a bottom surface portion of the second layer; and
providing a fourth bottom surface interior layer in contact with a bottom surface portion of the third layer and comprising a non-woven liquid permeable material having a planar, soft, cloth-like, body contact surface portion.

11. The method of claim 10 wherein the step of forming a second layer further comprises the step of providing a blended mixture of non-chlorine bleached wood pulp and cotton pulp having a substantially higher liquid urine absorption than wood pulp and located below a bottom surface of the underlying thin plastic sheet.

12. The method of claim 11 wherein the step of forming the second layer further comprises the step of mixing together separate sources of cotton pulp and non-chlorine bleached wood pulp material.

13. The method of claim 10 wherein the step of forming a second layer further comprises the step of providing a suberabsorbent mixture.

14. The method of claim 13 wherein the suberabsorbent mixture is a natural suberabsorbent mixture.

15. The method of claim 14 wherein the natural suberabsorbent mixture is a wheat based polysaccharide suberabsorbent mixture.

16. The method of claim 10 wherein the step of forming a second layer further comprises the step of providing a blended mixture of non-chlorine bleached wood pulp, cotton pulp, and a wheat based suberabsorbent mixture located below the bottom surface of the underlying thin plastic sheet.

17. The method of claim 10 further comprising the step of placing a layer comprising dioxin free tissue paper having a top surface thereof in contact with a bottom surface of an underlying thin plastic sheet and a bottom surface thereof in contact with a top surface of the second layer.

18. A disposable diaper comprising, in combinations: a multi-layered diaper assembly comprising:
a first top surface exterior layer assembly comprising:
a non-woven material having a planar, soft, cloth-like surface layer, the non-woven material surface layer;
an underlying thin, liquid impermeable plastic sheet;
a layer of dioxin free tissue paper having a top surface thereof in contact with a bottom surface of the underlying thin plastic sheet;
a second layer consisting of wood pulp;
a third layer having a top surface portion in contact with a bottom surface portion of the second layer; and
a fourth bottom surface interior layer in contact with a bottom surface portion of the third layer and comprising a non-woven liquid permeable material having a planar, soft, cloth-like body contact surface portion.

19. A disposable diaper in accordance with claim 18 wherein the second layer further comprises a blended mixture of dioxin free wood pulp and cotton pulp having a substantially higher liquid urine absorption than wood pulp and located below a bottom surface of the underlying thin plastic sheet.

20. A disposable diaper in accordance with claim 18 wherein the second layer further comprises a superabsorbent mixture.
